(19) United States
(12) Patent Application Publication
(10) Pub. No.: US 2023/0248657 A1
(43) Pub. Date: Aug. 10, 2023

(54) METHODS OF ADMINISTERING TASTE MASKED PHENYLBUTYRATE AND COMPOSITIONS THEREFOR

(71) Applicant: Acer Therapeutics Inc., Newton, MA (US)

(72) Inventor: D. Christopher SCHELLING, Bend, OR (US)

(21) Appl. No.: 18/014,665

(22) PCT Filed: Jul. 7, 2021
(86) PCT No.: PCT/US2021/040760

§ 371 Date: Jan. 5, 2023

Related U.S. Application Data
(60) Provisional application No. 63/065,272, filed on Aug. 13, 2020.
(60) Provisional application No. 63/048,892, filed on Jul. 7, 2020.

(51) Int. Cl. A61K 9/50; A61K 9/00; A61P 25/00; A61K 31/192

(52) U.S. Cl. CPC A61K 9/5078; A61K 9/0053; A61K 9/5026; A61P 25/00; A61K 31/192

(57) ABSTRACT

The present invention provides taste masked formulations of phenylbutyrate and methods of use in treatment nitrogen retention and other disorders. Formulations of the invention also reduced CNS adverse events and allow for longer consumption times.

### BACKGROUND OF THE INVENTION

Many inborn errors of metabolism, such as nitrogen retention disorders, are the result of defects of single genes that code for enzymes important for metabolism of certain substrates. The reduced activity of the enzymes results in accumulation of substrate to toxic levels which, in turn, leads to various symptoms depending on the substrate. Current treatments for nitrogen retention disorders are suboptimal. Accordingly, there is a need for new treatments.

### SUMMARY OF THE INVENTION

The present invention is directed to the administration of a therapeutically effective amount of taste masked phenylbutyrate, e.g., sodium phenylbutyrate (NaPB), to a subject in need thereof. In one embodiment, the administration is preprandial. In other embodiments, the dose is adjusted according to the time of day or ammonia levels, e.g., fasting ammonia levels.

In one embodiment, the invention provides a pharmaceutical composition for oral administration of phenylbutyrate including a particulate formulation including 50-75% by total weight of phenylbutyrate and 8-10% by total weight of a taste mask coating.

In some embodiments, the pharmaceutical composition is formulated as a plurality of spray-layered beads having a seed core, a drug layer comprising the phenylbutyrate, and the taste mask coating. In some embodiments, the seed core is 10-15% by total weight; the drug layer further includes a binder comprising 5-7% by total; the drug layer further includes a plasticizer being 0.5-1% by total weight; the taste mask coating includes a cationic copolymer of dimethylaminoethyl methacrylate, butyl methacrylate, and methyl methacrylate; and/or the taste mask coating includes an anti-tacking agent comprising 1-7% by total weight.

In some embodiments, the total weight of the taste masked coating is 8-10%. In some embodiments, the total weight of the taste masked coating is 15-18%. In some embodiments, the total weight of the taste masked coating is 21-25%.

In some embodiments, the total weight of the phenylbutyrate (e.g., NaPB) is about 65%. In some embodiments, the total weight of the phenylbutyrate (e.g., NaPB) is about 60%. In some embodiments, the total weight of the phenylbutyrate (e.g., NaPB) is about 55%.

In some embodiments, the particle size, e.g., DV90, is smaller than 500 μm, e.g., smaller than 400 μm or smaller than about 355 μm, as determined by sieving.

In one embodiment, the invention provides a method of administering taste masked phenylbutyrate to a subject having a nitrogen retention disorder by a) measuring a fasting ammonia level for the subject; b) comparing the fasting ammonia level to the upper limit of normal ammonia level; and c) administering the taste masked phenylbutyrate to the subject if the fasting ammonia level is greater than half the upper limit of normal ammonia level.

In some embodiments, the ammonia levels are blood ammonia levels or blood glutamine levels or the ammonia levels comprise breath ammonia levels. In some embodiments, the taste masked phenylbutyrate is a pharmaceutical composition as described herein.

In one embodiment, the invention provides a method of treating a nitrogen retention disorder or other disorder that can be treated with phenylbutyrate by administering a therapeutically effective amount of taste masked phenylbutyrate to a subject in need thereof, wherein administration is preprandial.

In some embodiments, the phenylbutyrate is administered at least 10 minutes prior to food. In some embodiments, the phenylbutyrate is an oral suspension that is consumed less than five minutes after constitution. In some embodiments, the taste masked phenylbutyrate is administered once or twice a day to the subject. In some embodiments, the administration occurs on more than one day. In some embodiments, the taste masked phenylbutyrate is a pharmaceutical composition as described herein.

In some embodiments, the taste masked phenylbutyrate of the invention is consumed in less than 10 minutes. In some embodiments, the taste masked phenylbutyrate is consumed in less than 5 minutes in a liquid suspension. In some embodiments, the taste masked phenylbutyrate is consumed in less than 4 minutes in a liquid suspension. In some embodiments, the taste masked phenylbutyrate is consumed in less than 3 minutes in a liquid suspension. In some embodiments, the taste masked phenylbutyrate is consumed in less than 2 minutes in a liquid suspension. In some embodiments, the taste masked phenylbutyrate is consumed in less than 1 minute in a liquid suspension.

In one embodiment, the invention provides a method of treating a nitrogen retention disorder or a disorder that can be treated with phenylbutyrate by administering a therapeutically effective amount of taste masked phenylbutyrate to a subject in need thereof, wherein the administering occurs optionally prior to a morning meal, prior to a midday meal, and prior to an evening meal, wherein the amount administered prior to the morning meal is less than the amount administered prior to the midday and evening meals or no phenylbutyrate is administered prior to the morning meal.

In another aspect, the invention provides a method of treating a nitrogen retention disorder or a disorder that can be treated with phenylbutyrate by administering a therapeutically effective amount of taste masked phenylbutyrate to a subject in need thereof and at risk for a CNS adverse event from phenylbutyrate, wherein the administration results in a reduction of the CNS adverse event, e.g., by at least about 10, 20, 30, 40, 50, 60, 70, or 75%.

In some embodiments, the taste masked phenylbutyrate is a pharmaceutical composition as described herein.

In some embodiments of any method described herein, the nitrogen retention disorder is hyperammonemia, urea cycle disorder, N-acetylglutamate synthase deficiency, carbamoyl phosphate synthetase deficiency, ornithine transcarbamoylase deficiency, citrullinemia type I, argininosuccinic aciduria, argininemia, hyperornithinemia, homocitrullinuria syndrome, or hepatic encephalopathy.

In some embodiments of any method described herein, the disorder being treated is maple syrup urine disease, pyruvate dehydrogenase complex deficiency, medium-chain acyl CoA dehydrogenase deficiency, ALS, X-linked adrenoleukodystrophy, progressive familial intrahepatic cholestasis, Parkinson's disease, Huntington's disease, DYT1 dystonia, malignant gliomas, oral squamous cell carcinoma, colon carcinoma, Burkitt lymphoma, primary acute myeloid leukemia, retinoblastoma, prostate cancer, medulloblastoma, radiation protection, or hepatocellular carcinoma.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” may be understood to permit standard variation as would be understood by those of ordinary skill in the art, e.g., ±10; and (v) where ranges are provided, endpoints are included.

As used herein, the term “administration” refers to the administration of a composition to a subject. Administration to an animal subject (e.g., to a human) may be by any appropriate route. For example, in some embodiments, administration may be bronchial (including by bronchial instillation), buccal, enteral, interdermal, intra-arterial, intradermal, intragastric (e.g., nasogastric intubation), intramedullary, intramuscular, intranasal, intraperitoneal, intrathecal, intravenous, intraventricular, mucosal, nasal, oral, rectal, subcutaneous, sublingual, topical, tracheal (including by intratracheal instillation), transdermal, vaginal, or vitreal.

The term “ammonia levels,” as used herein refers to the amount of ammonia/ammonium and ammonia carriers, e.g., glutamine.

An “effective amount” of a compound may vary according to factors such as the disease state, age, sex, and weight of the individual, and the ability of the compound to elicit the desired response. A therapeutically effective amount encompasses an amount in which any toxic or detrimental effects of the compound are outweighed by the therapeutically beneficial effects. An effective amount also encompasses an amount sufficient to confer benefit, e.g., clinical benefit.

The term “food,” as used herein, refers to solid food with sufficient bulk that it is not rapidly dissolved and absorbed in the stomach, such as food with low protein. For example, a meal, such as breakfast, lunch, or dinner.

The term “nitrogen retention disorder,” as used herein, refers to any disease associated with elevated blood nitrogen or ammonia levels, e.g., caused by mutations that result in a deficiency of one of the six enzymes in the urea cycle comprising N-acetylglutamate synthase (NAGS), carbamoyl phosphate synthase (CPS1), ornithine transcarbamylase (OTC), argininosuccinate synthase (AS), argininosuccinate lyase (AL), and arginase (ARG). Examples of nitrogen retention disorders include hyperammonemia, urea cycle disorders, N-acetylglutamate synthase deficiency, carbamoyl phosphate synthetase deficiency, ornithine transcarbamoylase deficiency, citrullinemia type I, argininosuccinic aciduria, argininemia, hyperornithinemia, hepatic encephalopathy, and homocitrullinuria syndrome.

The term “pharmaceutical composition,” as used herein, represents a composition containing a compound described herein formulated with a pharmaceutically acceptable excipient. Pharmaceutical compositions can be formulated, for example, for oral administration in unit dosage form (e.g., a tablet, capsule, caplet, gel cap, suspension, solution, or syrup); for topical administration (e.g., as a cream, gel, lotion, or ointment); for intravenous administration (e.g., as a sterile solution free of particulate emboli and in a solvent system suitable for intravenous use); or in any other pharmaceutically acceptable formulation.

The term “preprandial,” as used herein, means prior to eating food, e.g., at least 5 minutes prior to a meal, such as 10 minutes prior to a meal.

As used herein, the term “subject” or “patient” refers to any organism to which a compound or composition in accordance with the invention may be administered, e.g., for experimental, diagnostic, prophylactic, and/or therapeutic purposes. Typical subjects include any animal (e.g., mammals such as mice, rats, rabbits, non-human primates, and humans). A subject may seek or be in need of treatment, require treatment, be receiving treatment, be receiving treatment in the future, or be a human or animal who is under care by a trained professional for a particular disease or condition.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

### BRIEF DESCRIPTION OF THE DRAWINGS

FIG. 1 is a graph illustrating the mean plasma concentration of NaPB over time after administration of taste masked NaPB in a fed state and a fasted state.

FIG. 2 is a graph illustrating the mean plasma concentration of PAA over time after administration of taste masked NaPB in a fed state and a fasted state.

FIG. 3 is a set of graphs illustrating in silico safety and efficacy models of taste masked NaPB in fasted, virtual adult and child patients.

FIG. 4 is a graph illustrating the bioequivalence of the taste masked NaPB (circles) to Buphenyl® (triangles).

### DETAILED DESCRIPTION OF THE INVENTION

The present inventors have discovered improved administrations of taste masked phenylbutyrate, e.g., NaPB, for the treatment of nitrogen retention disorders and disorders that may be treated with phenylbutyrate. For example, the inventors have discovered that preprandial administration of taste masked phenylbutyrate (e.g., NaPB) results in an increase in the phenylbutyrate exposure relative to phenylbutyrate administered with food. In addition, the inventors have determined that blood ammonia levels are not static during the day, and phenylbutyrate (e.g., NaPB) may be administered so that therapeutic efficacy coincides with higher ammonia levels. The inventors have further discovered a method of adjusting the dose of phenylbutyrate (e.g., NaPB) based on measurement of ammonia levels.

These methods are advantageous in improving the dosing of phenylbutyrate (e.g., NaPB). As is known in the art, phenylbutyrate (e.g., NaPB) has an extremely unpalatable taste. Thus, per its FDA-approved label, sodium phenyl butyrate (i.e., BUPHENYL®) is to be administered with food. Without wishing to be bound by theory, administration of taste masked phenylbutyrate (e.g., NaPB) in a fasted state results in a higher Cmax, faster absorption, and/or increased bioavailability, allowing for a lower dose to achieve a therapeutic effect. In addition, taste masked phenylbutyrate (e.g., NaPB) allows for administration in the absence of food.

Again without wishing to be bound by theory, subjects store excess ammonia or glutamine in skeletal muscle. When that capacity is exceeded, blood ammonia levels increase. Typically, upon waking, the subject has low blood ammonia levels and may need less or even no phenylbutyrate (e.g., NaPB) at that time. As the subject consumes food and becomes active, blood ammonia levels may increase, and phenylbutyrate (e.g., NaPB) may be administered. Adjustment of the dosage of phenylbutyrate (e.g., NaPB) based on blood ammonia levels also allows for reducing the amount of phenylbutyrate (e.g., NaPB) needed for treatment. In particular, phenylbutyrate (e.g., NaPB) may be administered once or twice daily as compared to 3-6 times daily for BUPHENYL®.

### Methods of Treatment

Generally, the invention provides methods of treating disorders as described herein. In particular, a subject may be treated. In any method of the invention, taste masked phenylbutyrate (e.g., NaPB) may be administered as an oral suspension. Typically, the subject will consume the suspension in less than 5 minutes, e.g., between 0 and 4 minutes, after suspension. Administration may occur as long as treatment is required. Accordingly, treatment, e.g., preprandially, may occur for multiple days, e.g., daily for at least 1 week, 1 month, or 1 year. Administration, e.g., preprandial, may be once, twice, three times, or more per day, such as once or twice. Administration may be chronic, e.g., occurring over a period of at least one month, 6 months, 1 year, or lifetime. As discussed herein, multiple doses in a day may not be equal. In some embodiments, the subject is at least 2 years old, at least 6 years old, at least 8 years old, at least 9 years old, at least 12 years old, at least 15 years old, at least 18 years old, or at least 21 years old.

Preprandial Administration

The present invention features methods of administering taste masked phenylbutyrate (e.g., NaPB) preprandially. The taste masked phenylbutyrate (e.g., NaPB) can be administered prior to consuming food, e.g., a meal or snack. In some embodiments, the taste masked phenylbutyrate (e.g., NaPB) is administered at 10 least minutes prior to consumption of food. In other embodiments, the phenylbutyrate (e.g., NaPB) is administered at least 30 minutes after the most recent consumption of food, e.g., at least 1, 2, or 3 hours after.

In certain embodiments, the amount of taste masked phenylbutyrate (e.g., NaPB) administered preprandially is at least 5% less than that required to produce the same Cmax when phenylbutyrate (e.g., NaPB) is administered with food, e.g., up to about 10%, 15%, 20, 25%, 30%, 35%, 40%, 45%, 50%, 55%, 60%, 65%, 70%, or 75% less, such as about 20%, 25%, 30%, 35%, 40%, or 45% less.

Adjusted Dosing

The dosage of taste masked phenylbutyrate (e.g., NaPB) may also be adjusted based on blood ammonia levels of the subject. The blood ammonia level may be determined by assaying the blood directly or by proxy, e.g., breath ammonia level or urinary PAGN level.

For example, the dose of phenylbutyrate (e.g., NaPB) administered prior to the morning meal may not be necessary or may be less than the dosage administered prior to the midday and/or evening meal, e.g., 5-100% less, e.g., 50-75% less, 25-50% less, or 5-20% less. Accordingly, in certain embodiments, the invention provides for the administration of phenylbutyrate once or twice daily, e.g., prior to the midday and evening meal or prior to the evening meal. Thus, the amount of phenylbutyrate (e.g., NaPB) administered preprandially may be about one third or two thirds of the daily dose of NaPB administered with food.

The invention also provides methods for changing therapy from phenylbutyrate (e.g., NaPB or glycerol phenylbutyrate) to taste masked phenylbutyrate (e.g., NaPB). When a subject switches from non-taste masked NaPB (e.g., BUPHENYL®) or glycerol phenylbutyrate, the subject may be administered taste masked phenylbutyrate (e.g., NaPB) preprandially at a level that provides less active pharmaceutical ingredient, e.g., up to about 10%, 15%, 20%, 25%, 30%, 35%, 40%, 45%, 50%, 55%, 60%, 65%, 70%, or 75% less equivalents of phenylbutyrate, such as about 30% less. After initial administration, subjects may have their blood ammonia level, e.g., fasting, measured and compared to the upper limit of normal ammonia level or to the ammonia level of the subject's prior medication. If the ammonia level is greater than half the upper limit of normal ammonia level or greater than the level of the subject on the prior medication, the amount of taste masked phenylbutyrate (e.g., NaPB) administered to the subject is increased. The amount of taste masked phenylbutyrate (e.g., NaPB) may be maintained or reduced if the blood ammonia levels are less than half the upper limit of normal ammonia level or less than the level of the subject on the prior medication. In some embodiments, ammonia levels may be assessed by measuring blood ammonia levels, breath ammonia levels, or urinary glutamine levels. In certain embodiments, the upper level for normal (ULN) for blood ammonia may be in the range of about 26-64 μmol/L. In certain of these embodiments, the ULN for blood ammonia may be in the range of about 32-38 μmol/L or about 34-36 μmol/L, and in certain of these embodiments the ULN for blood ammonia is about 35 μmol/L. In certain embodiments, the ULN for blood ammonia may be in the range of about 50-65 μg/mL. In certain of these embodiments, the ULN for blood ammonia may be in the range of about 55-63 μg/mL or 57-61 μg/mL, and in certain of these embodiments the ULN for blood ammonia is about 59 μg/mL. For urinary glutamine, a concentration falling into the bottom 50^{th}, 45^{th}, 40^{th}, 35^{th}, 30^{th}, 25^{th}, or 20th percentile for age and body surface area (BSA) may be employed as the cutoff, see, e.g., Mokhtarani Molecular genetics and metabolism reports, 2015, 5:12-14, which is hereby incorporated by reference. For example, urinary glutamine <about 9000 μg/mL for <2 years old, <about 7000 μg/mL for >2 years with BSA≤1.3 m², and <about 5000 μg/mL for >2 years of age with BSA>1.3 m²) may be used as a cutoff. In certain embodiments, the amount of taste masked phenylbutyrate (e.g., NaPB) may be maintained or reduced if the urinary glutamine concentration is above the bottom 50^{th}, 45^{th}, 40^{th}, 35^{th}, 30^{th}, 25^{th}, or 20th percentile, e.g., above the 25^{th} percentile.

Reduced Adverse Events

The present invention features methods of administering taste masked phenylbutyrate (e.g., NaPB) to a subject at risk for an adverse event, e.g., a CNS adverse event, such as headache or dizziness. The present inventors have found that administration of the taste masked phenylbutyrate described herein results in fewer CNS adverse events compared to BUPHENYL®. Accordingly, the present methods allow for treatment with a reduction in the number of CNS adverse events or a reduced risk of a CNS adverse event. Risk for CNS adverse events may be determined by any suitable means, e.g., one or more CNS adverse events occurring after a period of one day, one week, or one month, after administration of BUPHENYL®. The methods may reduce the number of adverse events by 5-75%, e.g., by at least 5, 10, 15, 20, 25, 30, 35, 40, 45, or 50%, e.g., reduction by about 20-50%. Reduction may be determined based on a reduction in the number of events relative to a control (e.g., subject on BUPHENYL® or a calibrated control) over a period of time, e.g., one day, week, or month.

### Disorders

The present invention provides methods of treating nitrogen retention disorders, including, but not limited to, hyperammonemia, urea cycle disorder, N-acetylglutamate synthase deficiency, carbamoyl phosphate synthetase deficiency, ornithine transcarbamoylase deficiency, citrullinemia type I, argininosuccinic aciduria, argininemia, hyperornithinemia, homocitrullinuria syndrome, or hepatic encephalopathy. In treating these disorders, it is believed that phenylbutyrate, e.g., NaPB, is a prodrug for phenylacetate (PAA).

The invention also features methods of treating other disorders that can be treated by phenylbutyrate, e.g., NaPB, including, but not limited to, maple syrup urine disease, pyruvate dehydrogenase complex deficiency, medium-chain acyl CoA dehydrogenase deficiency, Amyotrophic Lateral Sclerosis (ALS) (alone or in combination with tauroursodeoxycholic acid), X-linked adrenoleukodystrophy, progressive familial intrahepatic cholestasis, Parkinson's disease, Huntington's disease, radiation protection, or DYT1 dystonia. Furthermore, the invention also features methods of treating various cancers including, but not limited to, malignant gliomas, oral squamous cell carcinoma, colon carcinoma, Burkitt lymphoma, primary acute myeloid leukemia, retinoblastoma, prostate cancer, medulloblastoma, or hepatocellular carcinoma. In treating disorders other than nitrogen retention disorders, phenylbutyrate, e.g., NaPB, is believed to be the active drug.

In certain embodiments, the administration of phenylbutyrate, e.g., preprandial taste masked phenylbutyrate, e.g., 5 g NaPB or equivalent, results in a Cmax of phenylbutyrate, e.g., NaPB, greater than 100 μg/mL, e.g., 100-500 μg/mL, 100-300 μg/mL, 200-300 μg/mL at least 125 μg/mL, 150 μg/mL, 175 μg/mL, or 200 μg/mL, e.g., up to 500 μg/mL or 300 μg/mL. In certain embodiments, the administration of phenylbutyrate, e.g., preprandial taste masked phenylbutyrate, e.g., 5 g NaPB or equivalent, results in an AUC₀₋₂₄ of phenylbutyrate, e.g., NaPB, approximately equal to that of 14.1 g NaPB that is not taste masked (BUPHNEYL®) administered in a fed stated TID.

### Taste Masked Phenylbutyrate

Phenylbutyrate (e.g., NaPB) can be formulated in a pharmaceutical composition to achieve enhanced exposure and palatability. Any pharmaceutically acceptable salt of phenylbutyrate may be employed, e.g., NaPB. Taste masked phenylbutyrate (e.g., NaPB) may be a layered composition, e.g., including a seed core (e.g., microcrystalline cellulose or sucrose), drug layer, and taste mask layer. A seal coat may also be included, e.g., about 2-5% of the total, such as about 3.2%. In particular, the taste masked phenylbutyrate (e.g., NaPB) is a particulate formulation, e.g., as described in WO 2017/160345, which is hereby incorporated by reference. In some embodiments, taste masked phenylbutyrate (e.g., NaPB) includes about 50-75% phenylbutyrate (e.g., NaPB), by total weight, such as about 55-65%, e.g., about 55%, 60%, or 65%, e.g., about 65.3%, 59.9%, or 55.3%. The taste masked coating may be present from about 5-25% by total weight, e.g., about 8-10%, such as about 9%, about 15-18%, such as about 17%, or 21-25% such as about 22.9%. An exemplary taste masked coating includes a cationic copolymer of dimethylaminoethyl methacrylate, butyl methacrylate, and methyl methacrylate (e.g., Eudragit E PO). Specific formulations of the invention are provided in the examples. Exemplary seed cores include those about 50-300 microns in diameters, e.g., about 50-250 microns, such as about 100-200 microns.

In certain embodiments, the taste masked formulation includes (i) a seed core, e.g., microcrystalline cellulose, e.g., about 10-17%, such as about 12-15%, such as about 14%; (ii) a drug layer including about 50-70% phenylbutyrate (e.g., NaPB), e.g., about 55-66%, such as about 65%; a binder, e.g., HPMC, such as HPMC E5, e.g., about 5-7%, such as about 6% or about 6.5%; and a plasticizer, e.g., PEG, such as PEG 6000, e.g., about 0.5-1%, such as about 0.7%; (iii) a seal coat including, e.g., HPMC, such as HPMC E5, e.g., about 2-4%, such as about 3%, and/or PEG, such as PEG 6000, e.g., about 0-1%, e.g., about 0.3%; and (iv) a taste mask coating, e.g., including a copolymer of dimethylaminoethyl methacrylate, butyl methacrylate, and methyl methacrylate (e.g., Eudragit E PO), e.g., about 5-17%, such as about 6-12%, e.g., about 6%; an optional anti-tacking agent, such as talc or magnesium stearate, e.g., about 2-7%, such as about 2.4%; and a plasticizer, e.g., PEG, such as PEG 6000, e.g., about 0.3-2%, e.g., about 0.5-2%, such as about 0.6%, where percentages are of the total formulation, e.g., excluding any components not in the particulates. The pharmaceutical composition may also include a glidant, e.g., silicon dioxide, about 0-1%, such as about 0.5%, in addition to the particulates.

In certain embodiments, the taste masked formulation includes (i) a seed core, e.g., microcrystalline cellulose, e.g., about 9-16%, such as about 12-14%, such as about 13%; (ii) a drug layer including about 50-70% phenylbutyrate (e.g., NaPB), e.g., about 55-66%, such as about 60%; a binder, e.g., HPMC, such as HPMC E5, e.g., about 5-7%, such as about 6%; and a plasticizer, e.g., PEG, such as PEG 6000, e.g., about 0.5-1%, such as about 0.6%; (iii) a seal coat including, e.g., HPMC, such as HPMC E5, e.g., about 2-4%, such as about 3%, and/or PEG, such as PEG 6000, e.g., about 0-1%, e.g., about 0.3%; and (iv) a taste mask coating, e.g., including a copolymer of dimethylaminoethyl methacrylate, butyl methacrylate, and methyl methacrylate (e.g., Eudragit E PO), e.g., about 5-17%, such as about 8-14%, e.g., about 11%; an optional anti-tacking agent, such as talc or magnesium stearate, e.g., about 2-7%, such as about 4.4%, and a plasticizer, e.g., PEG, such as PEG 6000, e.g., about 0.5-2%, such as about 1%, where percentages are of the total formulation, e.g., excluding any components not in the particulates. The pharmaceutical composition may also include a glidant, e.g., silicon dioxide, about 0-1%, such as about 0.5%, in addition to the particulates.

In certain embodiments, the taste masked formulation includes (i) a seed core, e.g., microcrystalline cellulose, e.g., about 10-17%, such as about 10-14%, such as about 12%; (ii) a drug layer including about 50-70% phenylbutyrate (e.g., NaPB), e.g., about 50-60%, such as about 55%; a binder, e.g., HPMC, such as HPMC E5, e.g., about 5-7%, such as about 5.5%; and a plasticizer, e.g., PEG, such as PEG 6000, e.g., about 0.5-1%, such as about 0.6%; (iii) a seal coat including, e.g., HPMC, such as HPMC E5, .g., about 2-4%, such as about 2.7%, and/or PEG, such as PEG 6000, e.g., about 0-1%, e.g., about 0.3%; and (iv) a taste mask coating, e.g., including a copolymer of dimethylaminoethyl methacrylate, butyl methacrylate, and methyl methacrylate (e.g., Eudragit E PO), e.g., about 5-17%, such as about 13-17%, e.g., about 15%; an optional anti-tacking agent, such as talc or magnesium stearate, e.g., about 2-7%, such as about 6%; and a plasticizer, e.g., PEG, such as PEG 6000, e.g., about 0.5-2.5%, such as about 1.5%, where percentages are of the total formulation, e.g., excluding any components not in the particulates. The pharmaceutical composition may also include a glidant, e.g., silicon dioxide, about 0-1%, such as about 0.5%, in addition to the particulates.

In one embodiment, the taste masked formulation includes (i) a seed core of microcrystalline cellulose at about 14%; (ii) a drug layer comprising about 65% NaPB; a binder HPMC E5, at about 6.5%; and a plasticizer PEG 6000, at about 0.7%; (iii) a seal coat comprising HPMC E5, at about 3.1%, and PEG 6000, at about 0.3%; and (iv) a taste mask coating of a copolymer of dimethylaminoethyl methacrylate, butyl methacrylate, and methyl methacrylate (e.g., Eudragit E PO) at about 6%, where percentages are of the total formulation, e.g., excluding any components not in the particulates. The taste masked coating may also include an anti-tacking agent, such as talc or magnesium stearate, e.g., about 2-7%, such as about 2.4%, and a plasticizer, e.g., PEG, such as PEG 6000, e.g., about 0.5-2%, such as about 0.6%, where percentages are of the total formulation. The pharmaceutical composition may also include a glidant, e.g., silicon dioxide, about 0-1%, such as about 0.5%, in addition to the particulates.

In one embodiment, the taste masked formulation includes (i) a seed core of microcrystalline cellulose at about 13%; (ii) a drug layer comprising about 60% NaPB; a binder HPMC E5, at about 6%; and a plasticizer PEG 6000, at about 0.6%; (iii) a seal coat comprising HPMC E5, at about 2.9%, and PEG 6000, at about 0.3%; and (iv) a taste mask coating of a copolymer of dimethylaminoethyl methacrylate, butyl methacrylate, and methyl methacrylate (e.g., Eudragit E PO) at about 11.1%, where percentages are of the total formulation, e.g., excluding any components not in the particulates. The taste masked coating may also include an anti-tacking agent, such as talc or magnesium stearate, e.g., about 2-7%, such as about 4.4%, and a plasticizer, e.g., PEG, such as PEG 6000, e.g., about 0.5-2%, such as about 1.1%, where percentages are of the total formulation. The pharmaceutical composition may also include a glidant, e.g., silicon dioxide, about 0-1%, such as about 0.5%, in addition to the particulates.

In one embodiment, the taste masked formulation includes (i) a seed core of microcrystalline cellulose at about 12%; (ii) a drug layer comprising about 55% NaPB; a binder HPMC E5, at about 5.5%; and a plasticizer PEG 6000, at about 0.6%; (iii) a seal coat comprising HPMC E5, at about 2.7%, and PEG 6000, at about 0.3%; and (iv) a taste mask coating of a copolymer of dimethylaminoethyl methacrylate, butyl methacrylate, and methyl methacrylate (e.g., Eudragit E PO) at about 15.3%, where percentages are of the total formulation, e.g., excluding any components not in the particulates. The taste masked coating may also include an anti-tacking agent, such as talc or magnesium stearate, e.g., about 2-7%, such as about 6.1% and a plasticizer, e.g., PEG, such as PEG 6000, e.g., about 0.5-2%, such as about 1.5%, where percentages are of the total formulation. The pharmaceutical composition may also include a glidant, e.g., silicon dioxide, about 0-1%, such as about 0.5%, in addition to the particulates.

In certain embodiments, the taste masked formulation includes (i) a seed core, e.g., microcrystalline cellulose, e.g., about 10-17%, such as about 12-15%, such as about 14%; (ii) a drug layer including about 50-70% phenylbutyrate (e.g., NaPB), e.g., about 55-66%, such as about 65%; a binder, e.g., HPMC, such as HPMC E5, e.g., about 5-7%, such as about 6% or about 6.5%; and a plasticizer, e.g., PEG, such as PEG 6000, e.g., about 0.5-1%, such as about 0.7%; and (iii) a taste mask coating, e.g., including a copolymer of dimethylaminoethyl methacrylate, butyl methacrylate, and methyl methacrylate (e.g., Eudragit E PO), e.g., about 5-17%, such as about 6-12%, e.g., about 6%; an optional anti-tacking agent, such as talc or magnesium stearate, e.g., about 2-7%, such as about 2.4%; and a plasticizer, e.g., PEG, such as PEG 6000, e.g., about 0.3-2%, e.g., about 0.5-2%, such as about 0.6%, where percentages are of the total formulation, e.g., excluding any components not in the particulates. The pharmaceutical composition may also include a glidant, e.g., silicon dioxide, about 0-1%, such as about 0.5%, in addition to the particulates.

In certain embodiments, the taste masked formulation includes (i) a seed core, e.g., microcrystalline cellulose, e.g., about 9-16%, such as about 12-14%, such as about 13%; (ii) a drug layer including about 50-70% phenylbutyrate (e.g., NaPB), e.g., about 55-66%, such as about 60%; a binder, e.g., HPMC, such as HPMC E5, e.g., about 5-7%, such as about 6%; and a plasticizer, e.g., PEG, such as PEG 6000, e.g., about 0.5-1%, such as about 0.6%; and (iii) a taste mask coating, e.g., including a copolymer of dimethylaminoethyl methacrylate, butyl methacrylate, and methyl methacrylate (e.g., Eudragit E PO), e.g., about 5-17%, such as about 8-14%, e.g., about 11%; an optional anti-tacking agent, such as talc or magnesium stearate, e.g., about 2-7%, such as about 4.4%, and a plasticizer, e.g., PEG, such as PEG 6000, e.g., about 0.5-2%, such as about 1%, where percentages are of the total formulation, e.g., excluding any components not in the particulates. The pharmaceutical composition may also include a glidant, e.g., silicon dioxide, about 0-1%, such as about 0.5%, in addition to the particulates.

In certain embodiments, the taste masked formulation includes (i) a seed core, e.g., microcrystalline cellulose, e.g., about 10-17%, such as about 10-14%, such as about 12%; (ii) a drug layer including about 50-70% phenylbutyrate (e.g., NaPB), e.g., about 50-60%, such as about 55%; a binder, e.g., HPMC, such as HPMC E5, e.g., about 5-7%, such as about 5.5%; and a plasticizer, e.g., PEG, such as PEG 6000, e.g., about 0.5-1%, such as about 0.6%; and (iii) a taste mask coating, e.g., including a copolymer of dimethylaminoethyl methacrylate, butyl methacrylate, and methyl methacrylate (e.g., Eudragit E PO), e.g., about 5-17%, such as about 13-17%, e.g., about 15%; an optional anti-tacking agent, such as talc or magnesium stearate, e.g., about 2-7%, such as about 6%; and a plasticizer, e.g., PEG, such as PEG 6000, e.g., about 0.5-2.5%, such as about 1.5%, where percentages are of the total formulation, e.g., excluding any components not in the particulates. The pharmaceutical composition may also include a glidant, e.g., silicon dioxide, about 0-1%, such as about 0.5%, in addition to the particulates.

Taste masked formulations may have a particle size such that the formulation does not feel gritty in mouth. For example, the DV90 of the particles may be less than 500 microns, e.g., less than about 450, 425, 400, 375, or 350 microns, e.g., as measured by laser diffractometry, dynamic light scattering, or sieving. In particular embodiments, the particles have a DV90 of less than about 400 microns, e.g., less than about 360 microns, as measured by sieving. In other embodiments, the particles are substantially spherical.

Exemplary salts of phenylbutyrate include sodium phenylbutyrate and potassium phenylbutyrate. Other salts of phenylbutyrate include aluminum, arginine, benzathine, calcium, chloroprocaine, choline, diethanolamine, ethanolamine, ethylenediamine, histidine, lithium, lysine, magnesium, meglumine, ornithine, potassium, procaine, triethylamine, and zinc salts. Any of these salts may be employed in the formulations of the invention. It will be understood that weight percentages based on NaPB may be adjusted taking into account differences in molecular weight of other counterions.

### Dosage and Formulation

Taste masked phenylbutyrate (e.g., NaPB) can be used in any suitable dose. Suitable doses and dosage regimens can be determined by conventional range finding techniques. Generally treatment is initiated with smaller dosages, which are less than the optimum dose. Thereafter, the dosage is increased by small increments until optimum effect under the circumstances is reached. For convenience, the total daily dosage may be divided and administered in portions during the day if desired. In proper doses and with suitable administration of certain compounds, the present invention provides for a wide range of responses. Typically, the dosages range from about 0-20 g/day. For example, in certain embodiments, phenylbutyrate (e.g., NaPB) may be administered from about 0-4 g/day, 5-7 g/day, 8-10 g/day, 11-13 g/day, 14-16 g/day, or 17-20 g/day.

In some embodiments, the dose is an amount sufficient to produce levels of phenylacetyl glutamine (PAGN), e.g., as measured in the urine of a subject, and/or sufficient to produce a therapeutic result and/or response, e.g., lower levels of ammonia measured in the blood and/or breath, or lower levels of glutamine measured in the urine.

In some embodiments, the treatment produces a serum Cmax of phenylacetate of less than 500 μg/mL, less than 450 μg/mL, less than 400 μg/mL, less than 350 μg/mL, less than 300 μg/mL, less than 200 μg/mL, or less than 100 μg/mL.

Formulations for oral use include particles containing the active ingredient(s) in a mixture with non-toxic pharmaceutically acceptable excipients, and such formulations are known to the skilled artisan Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, anti-adhesives (e.g., magnesium stearate, zinc stearate, stearic acid, silicas, hydrogenated vegetable oils, or talc), and anti-caking agents (e.g., colloidal silicon dioxide, microcrystalline cellulose, tricalcium phosphate, microcrystalline cellulose, magnesium stearate, sodium bicarbonate, sodium ferrocyanide, potassium ferrocyanide, calcium ferrocyanide, calcium phosphate, sodium silicate, colloidal silicon dioxide, calcium silicate, magnesium trisilicate, talcum powder, sodium aluminosilicate, potassium aluminum silicate, calcium aluminosilicate, bentonite, aluminum silicate, stearic acid, polydimethylsiloxane). Other pharmaceutically acceptable excipients can be colorants, flavoring agents, plasticizers, humectants, and buffering agents. In some embodiments, excipients (e.g., flavoring agents) are packaged with the composition. In some embodiments, excipients (e.g., flavorings) are packaged separately from the composition (e.g., are combined with the composition prior to administration).

The particles of the present formulations may be under 1000 μm, under 500 μm, under 450 μm, under 425 μm, under 400 μm, under 355 μm, under 350 μm, under 300 μm, under 200 μm, under 180 μm, under 100 μm. The particles of the present formulations may be between 100-1000 μm in size, more specifically between 100-500 μm in size, even more specifically between 250-400 μm in size. In one embodiment greater than 90%, more specifically greater than 95%, even more specifically greater than 99% of the pharmaceutical composition has a particle size of less than 500 μm. In one embodiment greater than 90%, more specifically greater than 95%, even more specifically greater than 99% of the pharmaceutical composition has a particle size of less than 425 μm. In one embodiment greater than 90%, more specifically greater than 95%, even more specifically greater than 98% of the pharmaceutical composition has a particle size of less than 355 μm.

Prior to administration, the taste masked formulation may be combined with a dosing vehicle that contains various pharmaceutically acceptable excipients such as viscosity modifiers, suspending or dispersing agents, flavoring agents, fragrances, dyes (colors), sweeteners, anti-caking agents, glidants (flow enhancers), and lubricants. The dosing vehicle may include a food starch, such as modified corn starch, or maltodextrin. The dosing vehicle may be mixed with the taste masked formulation and added to water and stirred, or the dosing vehicle may be added first to the water and mixed before addition of the taste masked formulation. Other palatable liquids may be used instead of water provided that the pH of the liquid is ≥6. In certain embodiments, a suspension includes water and a commercial thickening agent, e.g., Thick-It® Original or Thick-It®2, along with the taste masked phenylbutyrate. For example, approximately 15 g Thick-It® Original or Thick-It®2 powder is dissolved in 120 mL water by manually stirring. A weight-adjusted quantity of the present pharmaceutical composition containing the equivalent of 5 g sodium phenylbutyrate may be added to the mixture and stirred to suspend the powder, prior to administration.

### EXAMPLES

### Example 1—Food Effect on Concentration of Taste-Masked Phenylbutyrate

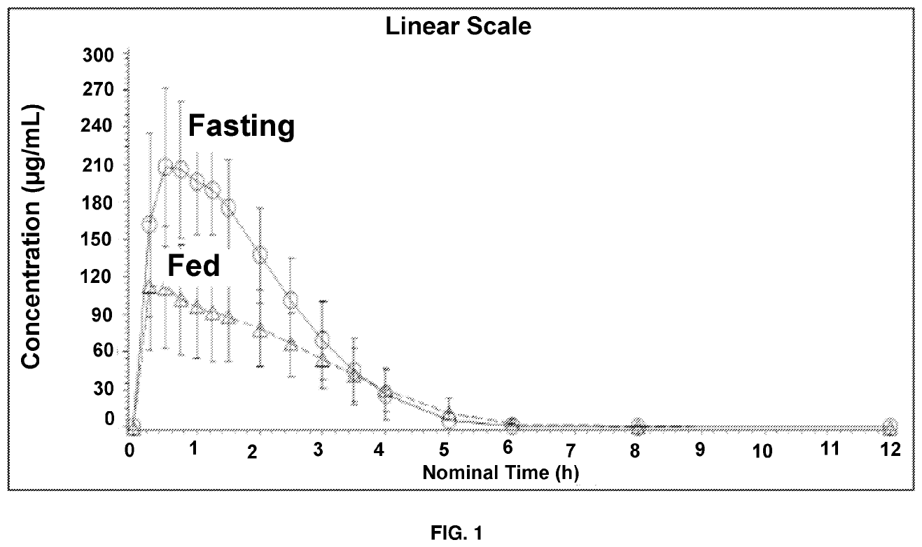

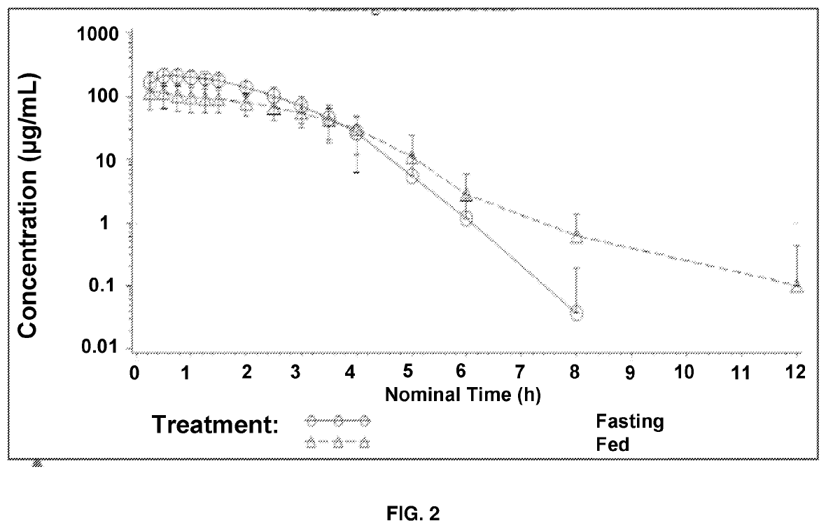

FIGS. 1 and 2 show the food effect on concentration of taste masked NaPB (5 g dose of NaPB) in healthy subjects. In FIG. 1, the Cmax of NaPB administered in the fasted state is approximately twice the Cmax of NaPB administered in the fed state. FIG. 2 shows the food effect on the concentration of PAA in healthy subjects. As shown, the Cmax of PAA after NaPB is administered in the fasted state is about 50% higher than Cmax of PAA after NaPB is administered in the fed state.

Table 1 shows multi-dose PK data of glycerol phenylbutyrate (GPB) vs. BUPHENYL (NaPB) vs. taste masked NaPB. When administered to a patient in a fasted state, 5 g, QD, of taste masked NaPB achieves a Cmax of phenylbutyrate almost three times that of 14.1 g NaPB (BUPHENYL®) administered TID to a patient in a fed state. In addition, the AUC of a single dose of taste masked NaPB, when administered to a patient in a fasted state, was about the same as the AUC of 14.1 g NaPB (BUPHENYL®) administered TID.

TABLE 1

| | Arithmetic Mean (CV %) | | |
|---|---|---|---|
| | | | Single-Dose |
| Plasma PK | Multi-Dose | | Taste masked |
| Parameters | GPB | NaPB | NaPB |
| Avg. Dose | 13.5 (TID, | 14.1 g (TID, | 5 g eq. (QD, |
| | fed) | fed) | fasted) |
| Plasma PBA | N = 44 | N = 44 | N = 36 |
| AUC₀₋₂₄ (μg*h/mL) | 443 | 508 | 510 |
| Cmax (μg/mL) | 51.9 | 80.9 | 229 |
| Tmax (h) | 8.0 | 3.0 | 0.5 |

### Example 2—Silico Modeling

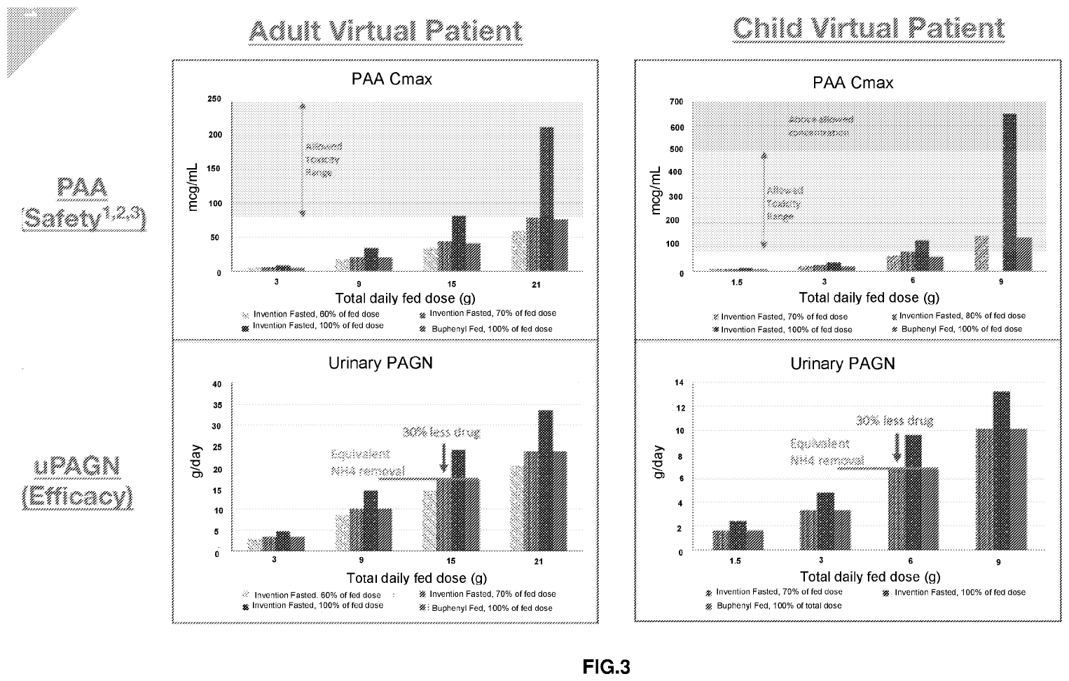

FIG. 3 shows in silico models of taste masked NaPB administered preprandially, which required approximately 30% less phenylbutyrate to achieve comparable therapeutic benefit compared to administration with food (Monteleone et al. J Clin Pharmacol. 2013, 53, 699 and Friedrich Pharmacometrics & Systems Pharmacology, 2016, 5, 45). The model also predicted a 43% increase in urinary PAGN levels (negative correlation with blood ammonia AUC).

### Example 3—Formulations

The taste masked phenylbutyrate (e.g., NaPB) may be a spray-layered multiparticulate powder formulation manufactured by fluid bed coating of a drug-containing layer onto a microcrystalline cellulose core, followed by a seal coat, and finally a taste-mask layer. The taste-masked multiparticulate powder may be then dry blended with a glidant.

Three multiparticulate formulations of the taste masked phenylbutyrate (e.g., NaPB) having three different taste-mask coating levels are shown in Tables 2-4, including the quantity of each component per nominal 5 g dose.

TABLE 2

| | Quality | | 1 | 2 | 3 |
|---|---|---|---|---|---|
| Component | Reference | Function | wt % | wt % | wt % |
| Seed core | | | | | |
| Microcrystalline | USP/NF, Ph. | Inert core | 13-16 | 12-15 | 11-13 |
| cellulose pellets | Eur., JP | | | | |
| Drug layer | | | | | |
| Sodium | USP, Ph. | Active | 60-70 | 55-65 | 5-60 |
| phenylbutyrate | Eur. | | | | |
| HPMC E5 | USP, Ph. | Binder | 6.5 | 6.0 | 5.5 |
| | Eur., JP | | | | |
| PEG 6000 | USP/NF, Ph. | Plasticizer | 0.5-1 | 0.5-1 | 0.5-1 |
| | Eur. | | | | |
| Seal coat | | | | | |
| HPMC E5 | USP, Ph. | Seal layer | 3.1 | 2.9 | 2.7 |
| | Eur., JP | | | | |
| PEG 6000 | USP/NF, Ph. | Seal layer | 0.3 | 0.3 | 0.3 |
| | Eur. | | | | |
| Taste-mask | | | | | |
| layer | | | | | |
| Eudragit E PO | NF, Ph. Eur., | Taste- | 6.0 | 11.1 | 15.3 |
| | JP | mask | | | |
| | | coating | | | |
| PEG 6000 | USP/NF, Ph. | Plasticizer | 0.6 | 1.1 | 1.5 |
| | Eur. | | | | |
| Talc | USP | Anti- | 2.4 | 4.4 | 6.1 |
| | | tacking | | | |
| | | agent | | | |
| Final blend | | | | | |
| Silicon dioxide | USP/NF, Ph. | Glidant | 0.5 | 0.5 | 0.5 |
| | Eur., JP | | | | |

| | | | |
|---|---|---|---|
| Total | 100.0 | 100.0 | 100.0 |

TABLE 3

| | | | Unit formula, 9.0% | | Unit formula, 16.6% | | Unit formula, 23.0% | |
|---|---|---|---|---|---|---|---|---|
| | | | taste-mask coat | | taste-mask coat | | taste-mask coat | |
| | | | weight | | weight | | weight | |
| | | | | mg per 5 | | mg per 5 | | mg per 5 |
| | Quality | | | g active | | g active | | g active |
| Component | Reference | Function | wt % | dose | wt % | dose | wt % | dose |

Seed core

| | | | | | | | | |
|---|---|---|---|---|---|---|---|---|
| Microcrystalline | USP/NF, Ph. | Inert core | 14.5 | 1106.6 | 13.3 | 1106.6 | 12.2 | 1106.6 |
| cellulose pellets | Eur., JP | | | | | | | |

Drug layer

| | | | | | | | | |
|---|---|---|---|---|---|---|---|---|
| Sodium | USP, Ph. | Active | 65.3 | 5,000.0 | 59.9 | 5,000.0 | 55.3 | 5,000.0 |
| phenylbutyrate | Eur. | | | | | | | |
| HPMC E5 | USP, Ph. | Binder | 6.5 | 500.0 | 6.0 | 500.0 | 5.5 | 500.0 |
| | Eur., JP | | | | | | | |
| PEG 6000 | USP/NF, Ph. | Plasticizer | 0.7 | 50.0 | 0.6 | 50.0 | 0.6 | 50.0 |
| | Eur. | | | | | | | |

Seal coat

| | | | | | | | | |
|---|---|---|---|---|---|---|---|---|
| HPMC E5 | USP, Ph. | Seal layer | 3.1 | 239.8 | 2.9 | 239.8 | 2.7 | 239.8 |
| | Eur., JP | | | | | | | |
| PEG 6000 | USP/NF, Ph. | Seal layer | 0.3 | 26.5 | 0.3 | 26.5 | 0.3 | 26.5 |
| | Eur. | | | | | | | |

Taste-mask layer

| | | | | | | | | |
|---|---|---|---|---|---|---|---|---|
| Eudragit E PO | NF, Ph. Eur., | Taste- | 6.0 | 461.5 | 11.1 | 923.1 | 15.3 | 1384.6 |
| | JP | mask | | | | | | |
| | | coating | | | | | | |
| PEG 6000 | USP/NF, Ph. | Plasticizer | 0.6 | 46.2 | 1.1 | 92.3 | 1.5 | 138.5 |
| | Eur. | | | | | | | |
| Talc | USP | Anti- | 2.4 | 184.6 | 4.4 | 369.2 | 6.1 | 553.8 |
| | | tacking | | | | | | |
| | | agent | | | | | | |

Final blend

| | | | | | | | | |
|---|---|---|---|---|---|---|---|---|
| Silicon dioxide | USP/NF, Ph. | Glidant | 0.5 | 38.3 | 0.5 | 41.7 | 0.5 | 45.2 |
| | Eur., JP | | | | | | | |

| | | | | | | |
|---|---|---|---|---|---|---|
| Total | 100.0 | 7,653.5 | 100.0 | 8,349.2 | 100.0 | 9,045.0 |

### Example 4—Particle Size Analysis

Approximately 100 g of the taste masked NaPB beads having 10% coat weight and 65.5% potency were used for each Bulk Density and Particle Size Analysis. A Ro-Tap® Particle Size Analyzer, fitted with U.S. Standard Hard Screens and a pan were used. A graduated cylinder was filled to the 100 mL mark with the beads. Approximately 20 g the present pharmaceutical composition was added to the screen and shaken for 5 minutes.

For the first analysis, the weight of the material used was 74.7 g, and the bulk density was 0.747 g/mL. Results are shown in Table 4.

TABLE 4

| | | | Tare | Net | | Screen |
|---|---|---|---|---|---|---|
| Sieve | Opening | Gross | Weight | Weight | % | Blinding |
| (Mesh#) | (μm) | Weight | (g) | (g) | Retained | (Y/N) |
| 35 | 500 | 336.1 | 336.1 | 0.0 | 0.0 | N |
| 40 | 425 | 337.2 | 337.2 | 0.0 | 0.0 | N |
| 45 | 355 | 337.4 | 337.1 | 0.3 | 1.5 | N |
| 50 | 300 | 340.9 | 326.1 | 14.8 | 74.0 | N |
| 60 | 250 | 314.4 | 309.5 | 4.9 | 24.5 | N |
| 80 | 180 | 306.8 | 306.8 | 0.0 | 0.0 | N |
| Pan | <180 | 373.6 | 373.6 | 0.0 | 0.0 | N |

| | | |
|---|---|---|
| Total | 20.0 | 100% |

For the second analysis, the weight of the material used was 76.5 g and the bulk density was 0.77 g/mL. Results are shown in Table 5.

TABLE 5

| | | | Tare | Net | | Screen |
|---|---|---|---|---|---|---|
| Sieve | Opening | Gross | Weight | Weight | % | Blinding |
| (Mesh#) | (μm) | Weight | (g) | (g) | Retained | (Y/N) |
| 35 | 500 | 336.1 | 336.1 | 0.0 | 0.0 | N |
| 40 | 425 | 327.2 | 337.2 | 0.0 | 0.0 | N |
| 45 | 355 | 337.4 | 337.0 | 0.4 | 2.0 | N |
| 50 | 300 | 342.9 | 325.9 | 17.0 | 85.0 | N |
| 60 | 250 | 312.1 | 309.5 | 2.6 | 13.0 | N |
| 80 | 180 | 307.0 | 307.0 | 0.0 | 0.0 | N |
| Pan | <180 | 373.6 | 373.6 | 0.0 | 0.0 | N |

| | | |
|---|---|---|
| Total | 20.0 | 100% |

For the third analysis results are shown in Table 6.

TABLE 6

| | | | Tare | Net | | Screen |
|---|---|---|---|---|---|---|
| Sieve | Opening | Gross | Weight | Weight | % | Blinding |
| (Mesh#) | (μm) | Weight | (g) | (g) | Retained | (Y/N) |
| 35 | 500 | 336.0 | 336.0 | 0.0 | 0.0 | N |
| 40 | 425 | 348.7 | 348.7 | 0.0 | 0.0 | N |
| 45 | 355 | 326.2 | 326.1 | 0.1 | 0.5 | N |
| 50 | 300 | 332.7 | 320.1 | 12.6 | 62.7 | N |
| 60 | 250 | 316.9 | 309.5 | 7.4 | 36.8 | N |
| 80 | 180 | 292.8 | 392.8 | 0.0 | 0.0 | N |
| Pan | <180 | 361.0 | 361.0 | 0.0 | 0.0 | N |

| | | |
|---|---|---|
| Total | 20.0 | 100% |

The results of the particle size analysis, determined via sieving, show that greater than 90%, more specifically greater than 95%, even more specifically greater than 98% of the pharmaceutical composition had a particle size of less than 355 μm.

### Example 5—Taste Analysis

The taste masked phenylbutyrate (e.g., NaPB) (denoted herein as “invention”) can be comfortably consumed over a longer period of time (i.e., much longer than 30 seconds), allowing for a more natural ingestion period.

The particles in this formulation include approximately 65% by weight sodium phenylbutyrate and approximately 9% by weight taste-mask coating (itself comprising 50-75% by weight of the polymer formed from dimethylaminoethyl methacrylate, butyl methacrylate, and methyl methacrylate (EUDRAGIT® E PO)) and are sized so that at least 90% of the particles are smaller than 500 μm.

Ten experienced pharmaceutical sensory panelists were screened and enrolled in the study. Panelists cleansed their palates with spring water and unsalted crackers. Each panelist was provided a disposable spoon, a 1 oz cup containing the API powder (Invention or BUPHENYL® (an uncoated formulation of sodium phenylbutyrate) equivalent to 208.3 mg sodium phenylbutyrate) and a syringe containing 5 mL of the dosing vehicle (water and Thick-it Original for Invention and water for BUPHENYL). Each panelist dispensed the dosing vehicle into the API containing cup, mixed with the disposable spoon for 30 seconds and waited for an additional 4.5 minutes for the 5-minute hold time preparation OR mixed for 30 seconds and waited an additional 9.5 minutes for the 10-minute hold time preparation. Starting at the same time, the panelists poured the contents of the cup directly in the mouth, swished contents around the oral cavity for 10 seconds and expectorated. During this time, the panelists independently evaluated and recorded the initial flavor characteristics. The panelists then independently evaluated and recorded the aftertaste characteristics at periodic intervals up to 20 minutes. The panelists recited their individual results and a preliminary Flavor Profile was generated for the sample. Steps 1 through 5 were repeated for a second evaluation of the sample using the preliminary Flavor Profile from Step 6 as a guide, with the panelists noting any necessary modifications. The panelists recited their individual results and a final Flavor Profile was developed for the sample.

The data is summarized in Table 7. Values above 1 are discernable by patients. Aversive attributes above the level of patient-perception (>1) are indicated in bold typeface.

TABLE 7

| Sample | | | | | | | | | | |
|---|---|---|---|---|---|---|---|---|---|---|
| | Hold | Basic | | | | | Mouthfeels | | | Score |
| | Time | Tastes | | Aromatic Off-Notes | | | | Tongue | Throat | (Lower is |
| Active | (min) | Bitter | Salt | Metallic | Soapy | Sulfite | Serum | Sting | Burn | Better) |
| Invention | 0 | 0.5 | 0.5 | 0.5 | — | — | — | — | — | 1.50 |
| Invention | 1 | 0.5 | 0.75 | 0.5 | — | — | — | — | — | 1.75 |
| Invention | 2 | 0.75 | 1.00 | 1.00 | — | 0.5 | 1.00 | 0.5 | — | 4.75 |
| Invention | 3 | 0.75 | 1.00 | 1.00 | — | 0.75 | 1.00 | 0.5 | — | 5.00 |
| Invention | 4 | 1.00 | 1.25 | 1.00 | — | 1.00 | 1.00 | 0.75 | — | 6.00 |
| Invention | 5 | 1.25 | 1.50 | 1.50 | — | 1.25 | 1.25 | 1.00 | — | 7.75 |
| Buphenyl ® | 1 | 2.00 | 2.00 | 1.75 | 1.00 | 2.00 | 1.50 | 1.00 | — | 11.25 |

The flavor quality of the taste masked phenylbutyrate (e.g., NaPB) was better than the flavor quality of Buphenyl® (an uncoated formulation of sodium phenylbutyrate) at all hold times. More importantly, the data suggest that the present taste masked phenylbutyrate (e.g., NaPB) can be consumed within 4-5 minutes while still maintaining acceptable taste-masking properties. This greatly increased duration of taste-masking is desirable and is very likely to increase patient compliance, especially among younger patients.

### Example 6—Clinical Trial Data

The same formulation of the taste masked phenylbutyrate used in Example 5 was tested in a clinical trial. The particles in this formulation include approximately 65% sodium phenylbutyrate and approximately 9% taste-mask coating and are sized so at least 90% of the particles are smaller than 500 μm. The study was a three arm study in 36 healthy subjects, cross-over design, where each subject received a single dose of the taste masked NaPB (denoted herein as “Invention”) (fasting), “Treatment A”; Buphenyl® (sodium phenylbutyrate) (fasting), “Treatment B”; or the taste masked NaPB (fed), “Treatment C”. All 36 subjects received each treatment (e.g., Treatment A, Treatment B, and Treatment C), and subjects were randomly assigned to 1 of 3 treatment sequences (1 [BAC], 2 [CBA], or 3 [ACB]), as follows in Table 8.

TABLE 8

| Sequence | Day 1 (Period 1) | Day 2 (Period 2) | Day 3 (Period 3) |
|---|---|---|---|
| 1 | B-5 g | A-5 g eq. | C-5 g eq. Invention |
| | BUPHENYL ® | Invention | fed |
| | fasting | fasting | |
| 2 | C-5 g | B-5 g | A-5 g Invention |
| | Invention fed | BUPHENYL ® | fasting |
| | | fasting | |
| 3 | A-5 g Invention | C-5 g | B-5 g BUPHENYL ® |
| | fasting | Invention fed | fasting |

Surprisingly, the formulations of the invention resulted in a significant reduction in treatment emergent adverse events (TEAEs) compared to Buphenyl®, as seen in Table 9.

TABLE 9

| | Treatment A | Treatment B | Treatment C |
|---|---|---|---|
| | Invention | BUPHENYL ® | Invention |
| | Fasting | Fasting | Fed |
| | (N = 36, healthy | (N = 36, healthy | (N = 36, healthy |
| | volunteer) | volunteers) | volunteers) |
| | n (%) [E] | n (%) [E] | n (%) [E] |
| AII TEAE | 7 (19.4) [15] | 14 (38.9) [28] | 5 (13.9) [6] |
| Nervous system | 6 (16.7) [9] | 13 (36.1) [20] | 2 (5.6) [3] |
| disorders | | | |
| Dizziness | 4 (11.1) [5] | 9 (25.0) [11] | 1 (2.8) [1] |
| Headache | 3 (8.3) [3] | 8 (22.2) [8] | 1 (2.8) [1] |
| Dysgeusia | 1 (2.8) [1] | 1 (2.8) [1] | 0 |
| Somnolence | 0 | 0 | 1 (2.8) [1] |

The number of subjects experiencing any TEAE was significantly lower for Treatment A (taste masked NaPB, fasting) vs. Treatment B (Buphenyl®, fasting). Comparatively, only 16.7% of patients who received Treatment A reported central nervous system (CNS) associated TEAEs, while 36.1% of patients who received Treatment B reported CNS associated TEAEs. More than twice the number of CNS disorder events (represented by [E]) were reported by patients when taking Buphenyl® compared with taste-masked NaPB (fasting), e.g., 20 events vs. 9 events, respectively. Furthermore, only 11.1% of patients who received Treatment A reported dizziness (5 events reported), while 25% of patients who received Treatment B reported dizziness (11 events reported, more than twice that of the taste-masked NaPB); only 8.3% of patients who received Treatment A reported headache (3 events reported), while 22.2% of patients who received Treatment B reported headache (8 events reported, more than twice that of taste-masked NaPB). The cross-over design of the study inherently provides each patient as his/her own matched control, which combined with the randomly assigned treatment sequence, provides strong support for the surprising outcome of reduced CNS associated TEAEs in patients taking the present taste-masked NaPB as compared with Buphenyl®.

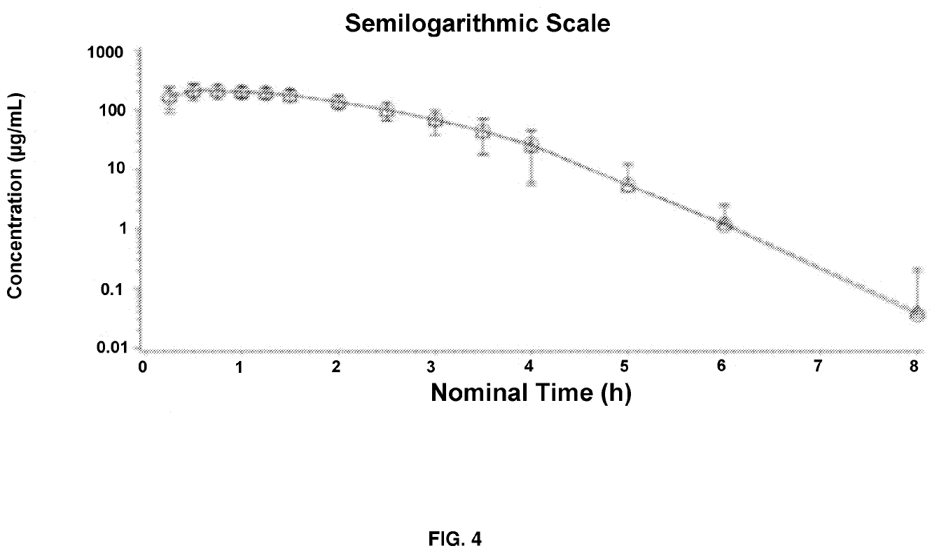

The reduction in CNS associated TEAEs is surprising because the present taste-masked NaPB rapidly releases sodium phenylbutyrate in the stomach, leading to a concentration of phenylbutyrate (PB) bioequivalent to that of Buphenyl®. The bioequivalence is evidenced in FIG. 4 showing the concentration of phenylbutyrate versus time for subjects administered a single oral dose (5 g) of each formulation. Accordingly, a reduced number of CNS associated TEAEs in taste-masked NaPB as compared to Buphenyl® is surprising due to their bioequivalence.

Other embodiments are in the claims.

What is claimed:

1. A pharmaceutical composition for oral administration of phenylbutyrate comprising 50-75% by total weight of phenylbutyrate and 8-25% by total weight of a taste mask coating.

2. The pharmaceutical composition of claim 1, wherein the pharmaceutical composition is formulated as a plurality of spray-layered beads comprising a seed core, a drug layer comprising the phenylbutyrate, and the taste mask coating.

3. The pharmaceutical composition of claim 2, wherein the seed core comprises 10-17% by total weight.

4. The pharmaceutical composition of any one of claims 2-3, wherein the drug layer further comprises a binder comprising 5-7% by total weight.

5. The pharmaceutical composition of any one of claims 2-4, wherein the drug layer further comprises a plasticizer comprising 0.5-1% by total weight.

6. The pharmaceutical composition of any one of claims 1-5, wherein the taste mask coating comprises a cationic copolymer of dimethylaminoethyl methacrylate, butyl methacrylate, and methyl methacrylate.

7. The pharmaceutical composition of any one of claims 1-6, wherein the taste mask coating comprises an anti-tacking agent comprising 1-7% by total weight.

8. The pharmaceutical composition of any one of claims 1-7, wherein at least 95% of the pharmaceutical composition has a particle size smaller than 355 μm as determined by sieving.

9. The pharmaceutical composition of any one of claims 1-8, wherein the total weight of the taste masked coating is 8-10%.

10. The pharmaceutical composition of any one of claims 1-8, wherein the total weight of the taste masked coating is 15-18%.

11. The pharmaceutical composition of any one of claims 1-8, wherein the total weight of the taste masked coating is 21-25%.

12. The pharmaceutical composition of any one of claims 1-11, wherein the total weight of the phenylbutyrate is about 65%.

13. The pharmaceutical composition of any one of claims 1-11, wherein the total weight of the phenylbutyrate is about 60%.

14. The pharmaceutical composition of any one of claims 1-11, wherein the total weight of the phenylbutyrate is about 55%.

15. A method of administering taste masked phenylbutyrate to a subject having a nitrogen retention disorder comprising:
a) measuring a fasting ammonia level for the subject;
b) comparing the fasting ammonia level to the upper limit of normal ammonia level; and
c) administering the taste masked phenylbutyrate to the subject if the fasting ammonia level is greater than half the upper limit of normal ammonia level.

16. The method of claim 15, wherein the ammonia levels comprise blood ammonia levels or blood glutamine levels.

17. The method of claim 15, wherein the ammonia levels comprise breath ammonia levels.

18. The method of any one of claims 15-17, wherein the taste masked phenylbutyrate is the pharmaceutical composition of any one of claims 1-14.

19. A method of treating a nitrogen retention disorder or other disorder that can be treated with phenylbutyrate comprising administering a therapeutically effective amount of taste masked phenylbutyrate to a subject in need thereof, wherein administration is preprandial.

20. The method of claim 19, wherein the phenylbutyrate is administered at least 10 minutes prior to food.

21. The method of claim 19, wherein the phenylbutyrate is an oral suspension that is consumed less than five minutes after constitution.

22. The method of claim 19, wherein the taste masked phenylbutyrate is administered once or twice a day to the subject.

23. The method of any one of claims 19-22, wherein the administration occurs on more than one day.

24. The method of any one of claims 19-23, wherein the taste masked phenylbutyrate is the pharmaceutical composition of any one of claims 1-14.

25. A method of treating a nitrogen retention disorder or a disorder that can be treated with phenylbutyrate comprising administering a therapeutically effective amount of taste masked phenylbutyrate to a subject in need thereof, wherein the administering occurs optionally prior to a morning meal, prior to a midday meal, and prior to an evening meal, wherein the amount administered prior to the morning meal is less than the amount administered prior to the midday and evening meals or no phenylbutyrate is administered prior to the morning meal.

26. The method of claim 25, wherein the taste masked phenylbutyrate is the pharmaceutical composition of any one of claims 1-14.

27. The method of any one of claims 15-26, wherein the nitrogen retention disorder comprises hyperammonemia, urea cycle disorder, N-acetylglutamate synthase deficiency, carbamoyl phosphate synthetase deficiency, ornithine transcarbamoylase deficiency, citrullinemia type I, argininosuccinic aciduria, argininemia, hyperornithinemia, homocitrullinuria syndrome, or hepatic encephalopathy.

28. The method of any one of claims 15-26, wherein the disorder being treated is maple syrup urine disease, pyruvate dehydrogenase complex deficiency, medium-chain acyl CoA dehydrogenase deficiency, ALS, X-linked adrenoleukodystrophy, progressive familial intrahepatic cholestasis, Parkinson's disease, Huntington's disease, DYT1 dystonia, malignant gliomas, oral squamous cell carcinoma, colon carcinoma, Burkitt lymphoma, primary acute myeloid leukemia, retinoblastoma, prostate cancer, medulloblastoma, radiation protection, or hepatocellular carcinoma.

29. A method of treating a nitrogen retention disorder or a disorder that can be treated with phenylbutyrate comprising administering a therapeutically effective amount of taste masked phenylbutyrate to a subject in need thereof and at risk for a CNS adverse event from phenylbutyrate, wherein the administration results in a reduction of the CNS adverse event.

30. The method of claim 29, wherein the taste masked phenylbutyrate is the pharmaceutical composition of any one of claims 1-14.

31. The method of any one of claims 15-30, wherein the taste masked phenylbutyrate is consumed in less than 4 minutes in a liquid suspension.

32. The method of any one of claims 15-30, wherein the taste masked phenylbutyrate is consumed in less than 5 minutes in a liquid suspension.
